(19) United States
(12) Patent Application Publication
(10) Pub. No.: US 2024/0157088 A1
(43) Pub. Date: May 16, 2024

(54) CATHETER

(71) Applicant: TERUMO KABUSHIKI KAISHA, Tokyo (JP)

(72) Inventors: Ryo OKAMURA, Fujinomiya (JP); Tsutomu SUGIKI, Hadano (JP); Gen OMURA, Fujinomiya (JP)

(73) Assignee: TERUMO KABUSHIKI KAISHA, Tokyo (JP)

(21) Appl. No.: 18/421,358

(22) Filed: Jan. 24, 2024

Related U.S. Application Data
(63) Continuation of application No. PCT/JP2022/032115, filed on Aug. 25, 2022.

(30) Foreign Application Priority Data
Aug. 26, 2021 (JP) 2021-138213

(51) Int. Cl. A61M 25/00; A61M 25/01

(52) U.S. Cl. CPC A61M 25/005; A61M 25/0012; A61M 25/0045; A61M 25/0108

(57) ABSTRACT

A catheter includes: a catheter body wall having an elongated tubular shape; and at least one wire provided in the catheter body wall, the at least one wire having at least one groove.

### CROSS-REFERENCES TO RELATED APPLICATIONS

This application is a continuation of International Patent Application No. PCT/JP2022/032115 filed on Aug. 25, 2022, which claims priority to Japanese Patent Application No. 2021-138213 filed on Aug. 26, 2021, the entire content of both of which is incorporated herein by reference.

### TECHNOLOGICAL FIELD

The present disclosure generally relates to a catheter.

### BACKGROUND DISCUSSION

A catheter (see, for example, WO 2018/043554) such as a angiography catheter, a guiding catheter, or a microcatheter has an elongated tubular catheter body wall and at least one wire provided in the catheter body wall and constituting a coil body, a braided body, or the like as a radiopaque marker, a reinforcing body, or the like.

### SUMMARY

The catheter needs to have flexibility that is appropriately set while ensuring pushability.

In view of this, the catheter disclosed here is configured to have flexibility that is easily set.

One aspect of the present disclosure provides a catheter including: a catheter body wall having an elongated tubular shape; and at least one wire provided in the catheter body wall, the at least one wire having at least one groove.

As one embodiment of the present disclosure, in the catheter, the at least one groove is provided only in a distal portion of the catheter.

As one embodiment of the present disclosure, in the catheter, the at least one groove has a pitch that decreases from a proximal side toward a distal side of the catheter.

As one embodiment of the present disclosure, in the catheter, the at least one groove has a width or a depth that increases from the proximal side toward the distal side of the catheter.

As one embodiment of the present disclosure, in the catheter, the at least one wire forms a radiopaque marker or a reinforcing body.

As one embodiment of the present disclosure, in the catheter, the at least one wire is provided in the catheter body wall over the entire circumference.

As one embodiment of the present disclosure, in the catheter, the at least one wire forms a coil body or a braided body.

As one embodiment of the present disclosure, in the catheter, the at least one groove is provided in a section of the at least one wire extending along the entire circumference of the catheter body wall.

As one embodiment of the present disclosure, in the catheter, the at least one groove is provided in a section of the at least one wire extending in a second direction perpendicular to both an axial direction of the catheter body wall and a first direction perpendicular to the axial direction of the catheter body wall without being provided in a section of the at least one wire extending in the first direction.

As one embodiment of the present disclosure, in the catheter, the at least one groove is provided in only both surfaces or in only one surface of the at least one wire in the second direction.

As one embodiment of the present disclosure, in the catheter, the at least one groove is provided in only both surfaces or in only one surface of the at least one wire in the axial direction of the catheter body wall.

As one embodiment of the present disclosure, in the catheter, the at least one groove extends in the axial direction of the catheter body wall.

As one embodiment of the present disclosure, in the catheter, the at least one groove is provided only in a section of the at least one wire located outside the catheter body wall in a radial direction.

As one embodiment of the present disclosure, in the catheter, the at least one groove is provided helically or annularly in an outer peripheral surface of the at least one wire.

As one embodiment of the present disclosure, a method for manufacturing the catheter includes forming the at least one groove by irradiating the at least one wire with a laser beam in a state where the at least one wire is provided in the catheter body wall.

According to another aspect of the disclosure here, a catheter comprises: a catheter body wall having an elongated tubular shape; and at least one wire in the catheter body wall. The catheter body wall is comprised of an inner layer and an outer layer, with the outer layer of the catheter body wall being positioned radially outwardly of the inner layer of the catheter body wall. The least one wire is positioned radially inwardly of the outer peripheral surface of the catheter body wall and radially outwardly of the inner peripheral surface of the catheter body wall. The at least one wire is provides with at least one groove, and a part of the outer layer of the catheter body wall is located in at least a portion of the at least one groove of the at least one wire.

The present disclosure can provide a catheter having flexibility that is easily set.

### BRIEF DESCRIPTION OF THE DRAWINGS

FIG. 1 is a side view of a catheter according to one embodiment.

FIG. 2 is a partial cross-sectional side view taken along the section line II-II in FIG. 1.

FIG. 3 is a cross-sectional view taken along the section line III-Ill in FIG. 2.

FIG. 4 is a side view illustrating a modification of a reinforcing body illustrated in FIG. 1.

FIG. 5 is a side view illustrating a modification of a groove portion illustrated in FIG. 1.

FIG. 6 is a side view illustrating a modification of the groove portion illustrated in FIG. 1.

FIG. 7 is a side view illustrating a modification of the groove portion illustrated in FIG. 1.

FIG. 8 is a side view illustrating a modification of the groove portion illustrated in FIG. 1.

### DETAILED DESCRIPTION

An embodiment of catheter, representing an example of the new catheter disclosed here, will be described in detail below with reference to the drawings.

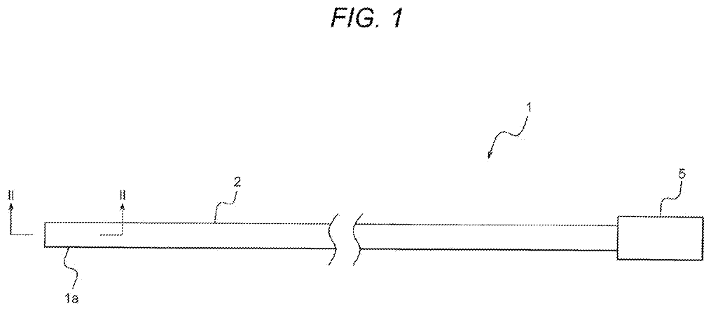

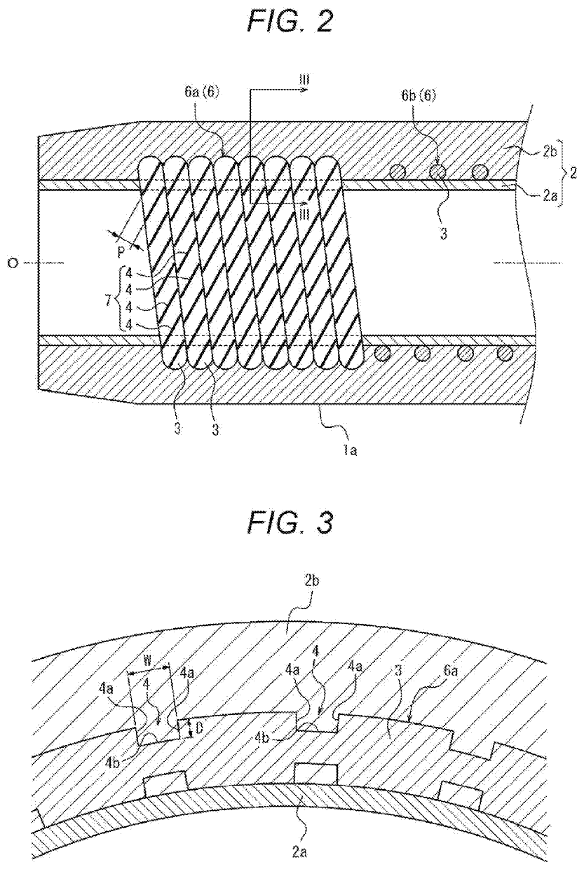

As illustrated in FIGS. 1 to 3, a catheter 1 according to one embodiment of the present disclosure includes a catheter body wall 2 having an elongated tubular shape with an axis O as a center (center axis) and at least one wire 3 provided in the catheter body wall 2, the at least one wire 3 having at least one groove 4. The catheter 1 may be qn angiography catheter, a guiding catheter, a microcatheter, or the like.

More specifically, the catheter 1 includes the catheter body wall 2, a hub 5, a first wire portion 6a, a second wire portion 6b, and a groove portion 7. The hub 5 is connected to the proximal end of the catheter body wall 2. During introduction of the catheter 1 into a lumen in a living body, a front end of the catheter 1 is referred to as a distal end, and an end of the catheter 1 opposite to the front end is referred to as a proximal end in the present embodiment. The hub 5 has a tubular shape with the axis O as a center (center axis), and a lumen of the hub 5 communicates with a lumen of the catheter body wall 2.

The catheter body wall 2 has an inner layer 2a and an outer layer 2b. The inner layer 2a and the outer layer 2b each have an elongated tubular shape with the axis O as a center (center axis), and the outer layer 2b is provided outside the inner layer 2a in the radial direction. Each of the inner layer 2a and the outer layer 2b is formed of a synthetic resin material. A material for forming each of the inner layer 2a and the outer layer 2b can be appropriately set according to the application of the catheter 1 or the like. Each of the first wire portion 6a and the second wire portion 6b is provided between the inner layer 2a and the outer layer 2b. Each of the first wire portion 6a and the second wire portion 6b is embedded in the outer layer 2b on the outside of the inner layer 2a in the radial direction.

The first wire portion 6a includes two wires 3 and is a double helical coil body (two wires helically wound in a coil) with the axis O as a center. That is, the first wire portion 6a is provided over the entire circumference in the catheter body wall 2 so that the first wire portion 6a has a circumferential extent of at least 360°. The first wire portion 6a forms a radiopaque marker. That is, the first wire portion 6a is formed of a material having low X-ray transparency, such as Pt or Au. The cross-sectional shape (shape in a cross section perpendicular to the axial direction) of each wire 3 constituting the first wire portion 6a is circular. The first wire portion 6a is provided only at a distal portion 1a of the catheter 1. The first wire portion 6a is provided on the distal side with respect to the second wire portion 6b.

The second wire portion 6b includes one wire 3 and is a single helical coil body with the axis O as a center. That is, the second wire portion 6b is provided over the entire circumference in the catheter body wall 2. The second wire portion 6b constitutes a reinforcing body. That is, the second wire portion 6b is formed of a material having high rigidity such as metal. The wire 3 constituting the second wire portion 6b has a circular cross-sectional shape. The second wire portion 6b is provided over the entire circumference in the catheter body wall 2 except for the distal portion of the catheter body wall 2.

The outer peripheral surface of each wire 3 constituting the first wire portion 6a has a double helical groove 4 including two grooves 4. Therefore, the first wire portion 6a has a groove portion 7 constituted by the four grooves 4. Each groove 4 extends helically along the axial direction (longitudinal direction) of the wire 3. Each groove 4 has two side surfaces 4a facing each other and a bottom surface 4b connecting the side surfaces 4a. The material forming the outer layer 2b enters each groove 4, achieving strong adhesion strength between the outer layer 2b and the first wire portion 6a.

Each groove 4 has a constant width W (distance between both side surfaces 4a) and a constant depth D (depth D of the bottom surface 4b). The groove portion 7 forms a predetermined pattern in which a part of the groove 4 is arranged at a predetermined pitch P in the width direction of the groove 4. The pitch P of the groove portion 7 is constant.

Each groove 4 is provided over the entire length of the wire 3 in the axial direction. Therefore, the groove portion 7 is provided in a section of the first wire portion 6a extending along the entire circumference with the axis O as a center.

A method for forming each groove 4 is not particularly limited. For example, each groove 4 can be formed by cutting the outer peripheral surface of the wire 3 with a laser or the like emitted perpendicularly to the axial direction, as the wire 3, which is linearly extended before being formed into a coil shape, is fed in the axial direction while being rotated in the circumferential direction.

The outer peripheral surface of each wire 3 constituting the second wire portion 6b does not have the groove 4. That is, the groove 4 is provided only in the first wire portion 6a. Therefore, the groove 4 is provided only at the distal portion 1a of the catheter 1.

According to the present embodiment, the groove portion 7 is provided, whereby the flexibility can be enhanced while ensuring the X-ray contrast properties of the first wire portion 6a. In addition, the flexibility of the distal portion 1a of the catheter 1 can be easily set by appropriately setting the width W, the depth D, and the pitch P of the grooves 4 of the groove portion 7.

The first wire portion 6a may be a coil body having a helical structure other than the double helical structure, and may be, for example, a single helical coil body or a coil body having a multiple (triple or more) helical structure. The cross section of each wire 3 constituting the first wire portion 6a may have a shape other than a circular shape such as an elliptical shape, a rectangular shape, a square shape, or a polygonal shape.

The second wire portion 6b may be a coil body having a helical structure other than the single helical structure, and may be, for example, a double helical coil body or a coil body having a multiple (triple or more) helical structure. The cross section of each wire 3 constituting the second wire portion 6b may have a shape other than a circular shape such as an elliptical shape, a rectangular shape, a square shape, or a polygonal shape.

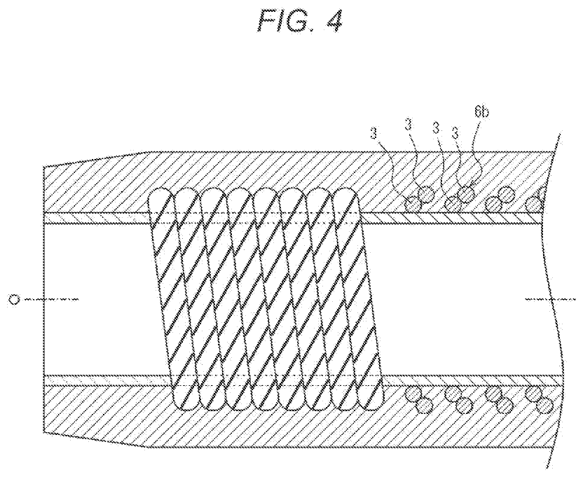

The second wire portion 6b may be a braided body formed in a tubular shape by braiding a plurality of wires 3, instead of the coil body. For example, the second wire portion 6b may be a mesh tubular braided body as a reinforcing body also referred to as a braid as in a modification illustrated in FIG. 4. In the modification illustrated in FIG. 4, the cross-sectional shape of each wire 3 constituting the braided body is circular. However, the cross section of each wire 3 constituting the braided body may have a shape other than a circular shape such as an elliptical shape, a rectangular shape, a square shape, or a polygonal shape.

The first wire portion 6a may not be provided on the distal side with respect to the second wire portion 6b. For example, a distal portion of the second wire portion 6b may be provided between the first wire portion 6a and the inner layer 2a.

The groove 4 formed in the outer peripheral surface of each wire 3 constituting the first wire portion 6a may have a helical structure other than the double helical structure, and may have, for example, a single helical structure or a multiple (triple or more) helical structure.

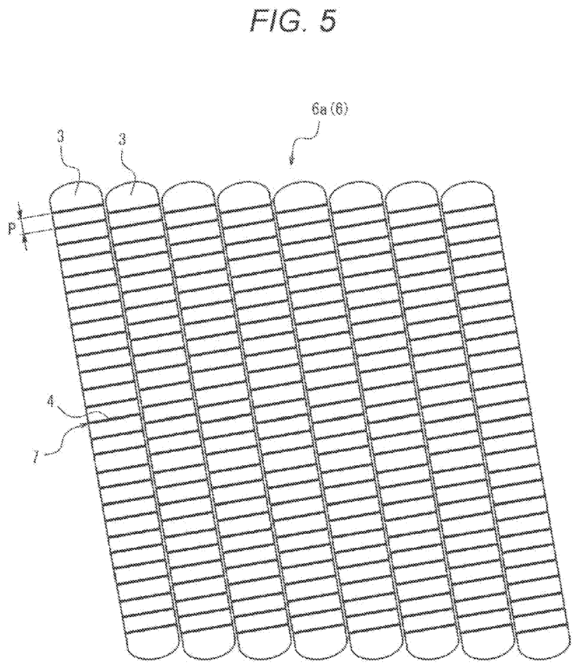

Each groove 4 constituting the groove portion 7 may be provided annularly instead of being provided helically. For example, as in a modification illustrated in FIG. 5, the outer peripheral surface of each wire 3 constituting the first wire portion 6a may have a plurality of annular grooves 4 arranged at a predetermined pitch P in the axial direction of the wire 3. In FIG. 5, only one groove 4 is denoted by reference numeral “4”, though the drawing figure depicts a plurality of such grooves.

The groove portion 7 may be provided not only in the first wire portion 6a but also in the second wire portion 6b. In this case, each groove 4 constituting the groove portion 7 of the second wire portion 6b may be provided helically or annularly.

The first wire portion 6a having the groove portion 7 may be provided without providing the second wire portion 6b. The second wire portion 6b having the groove portion 7 may be provided without providing the first wire portion 6a. With such a configuration, it is possible to achieve appropriate flexibility without impairing the reinforcing effect, for example, kink resistance, by the second wire portion 6b.

Not all the wires 3 constituting the first wire portion 6a but only some of the wires 3 may have at least one groove 4. Not all the wires 3 constituting the second wire portion 6b but only some of the wires 3 may have at least one groove 4.

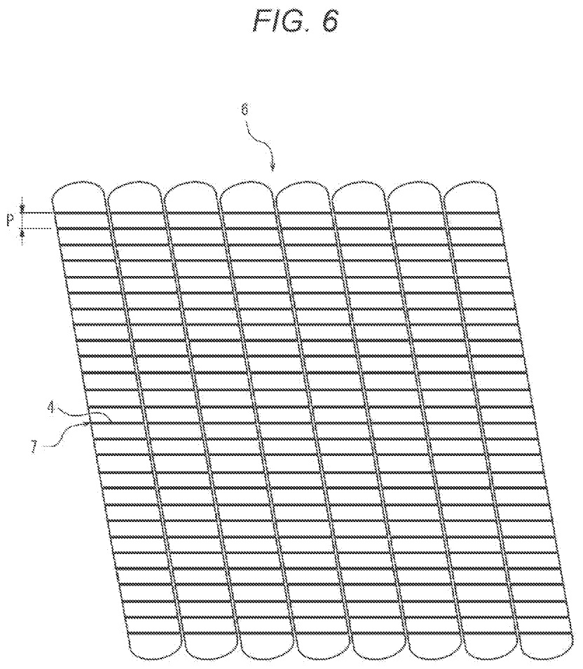

Hereinafter, at least one of the first wire portion 6a and the second wire portion 6b is referred to as wire portion 6. Each groove 4 constituting the groove portion 7 provided in the wire portion 6 may be provided in a shape other than the annular shape and the helical shape. For example, as in a modification illustrated in FIG. 6, each groove 4 constituting the groove portion 7 provided in the wire portion 6 may extend along the axial direction (direction along the axis O) of the catheter body wall 2 so that the grooves 4 are parallel to the central axis O of the catheter body wall 2. In this case, each groove 4 may be provided only in a section of the wire portion 6 facing toward the outside of the catheter body wall 2 (outside of the axis O) in the radial direction. In FIG. 6, only one groove 4 is denoted by reference numeral “4”, though the drawing figure depicts a plurality of such grooves. According to such a configuration, the groove portion 7 can be easily formed by an irradiation step of forming at least one groove 4 by irradiating the wire portion 6 (at least one wire 3) with a laser beam in a state where the wire portion 6 is provided in the catheter body wall 2. The wavelength of the laser beam used in the irradiation step is set to a wavelength at which the wire portion 6 made of metal or the like easily absorbs heat and the catheter body wall 2 made of synthetic resin or the like hardly absorbs heat. The irradiation step may be performed after the wire portion 6 is provided on the outer surface of the inner layer 2a of the catheter body wall 2 and before the outer layer 2b is provided.

Each groove 4 constituting the groove portion 7 provided in the wire portion 6 may extend in the axial direction of the catheter body wall 2 or may extend in other directions. Each groove 4 constituting the groove portion 7 provided in the wire portion 6 may be provided in a section of the wire portion 6 facing toward the inside of the catheter body wall 2 in the radial direction in addition to or instead of being provided in the section of the wire portion 6 facing toward the outside of the catheter body wall 2 in the radial direction. The groove portion 7 provided in the wire portion 6 may be provided on both of or one of the surfaces of at least one wire 3 in the axial direction of the catheter body wall 2.

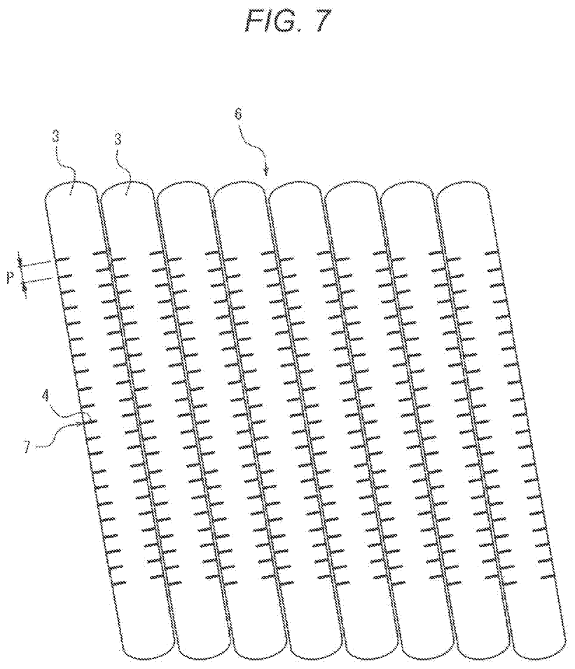

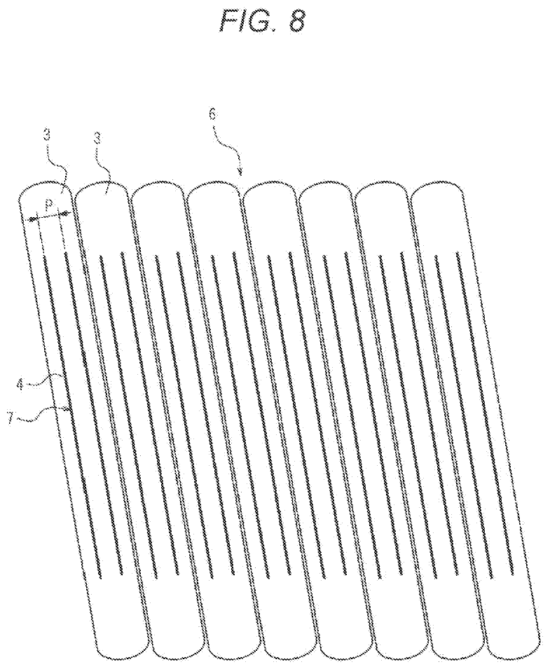

The description above explains that the groove portion 7 may be provided in the section of the wire portion 6 extending along the entire circumference of the catheter body wall 2. The groove portion 7 is not limited to extending along the entire circumference of the catheter body wall 2. For example, as shown in the modifications depicted in FIG. 7 and FIG. 8, the groove portion 7 may be provided in a section of the wire portion 6 extending in a second direction (direction perpendicular to the page (plane of the paper) of FIG. 7 or FIG. 8) that is perpendicular to both the axial direction of the catheter body wall 2 (left and right direction in FIGS. 7 and 8) and a first direction (vertical or up/down direction in FIGS. 7 and 8) that is perpendicular to the axial direction of the catheter body wall 2. At the same time, the groove portion 7 is not provided in a section of the wire portion 6 extending in the first direction (vertical or up/down direction in FIG. 7 and FIG. 8) as also shown by way of example in the modifications illustrated in FIG. 7 and FIG. 8. In FIG. 7 or FIG. 8, only one groove 4 is denoted by a reference sign. According to such a configuration, the flexibility in a specific direction can be enhanced while ensuring the pushability of the wire portion 6.

The groove portion 7 provided in the section of the wire portion 6 extending in the second direction may be provided in only both surfaces of at least one wire 3 in the axial direction of the catheter body wall 2 as in the modification illustrated in FIG. 7, for example. That is, the groove portion 7 provided in the section of the wire portion 6 extending in the second direction may be provided in the surfaces facing to the left and to the right as seen with reference to FIG. 7, and may be configured so that grooves of limited circumferential extent (extending less than 360° around the circumference of the at least one wire 3) are spaced apart from one another along the axial extent of the wire 3 as also shown in FIG. 7. Alternatively, the groove portion 7 provided in the section of the wire portion 6 extending in the second direction may be provided in only one surface of at least one wire 3 in the axial direction of the catheter body wall 2. According to such a configuration, the flexibility in the first direction can be enhanced while ensuring the pushability of the wire portion 6.

The groove portion 7 provided in the section of the wire portion 6 extending in the second direction may be provided on only both surfaces or on only one surface of at least one wire 3 in the second direction as in the modification illustrated in FIG. 8, for example. Thus, as shown in FIG. 8, the groove portion 7 may be configured so that grooves extend along the axial extent of the wire 3 in a limited axial extent and are spaced apart from one another in an axial direction of the catheter body wall 2. According to such a configuration, the flexibility in the second direction can be enhanced while ensuring the pushability of the wire portion 6.

The groove portion 7 provided in the wire portion 6 is not limited to being configured so that the groove has a constant width W, a constant depth D, and/or a constant pitch P. For example, in the groove portion 7 provided in the wire portion 6, the pitch P of the grooves 4 may decrease from the proximal side toward the distal side of the catheter 1, or the width W or the depth D of the grooves 4 may increase from the proximal side toward the distal side of the catheter 1. According to such a configuration, the flexibility of the distal portion 1a of the catheter 1 can be efficiently enhanced while ensuring the pushability of the wire portion 6.

The present disclosure is not limited to the embodiments described above and may be modified in various manners without departing from the gist of the present disclosure.

Thus, the catheter 1 according to the above-described embodiments can be variously changed as described below, for example.

The catheter 1 according to the above embodiments can be variously changed, as long as it includes the catheter body wall 2 having an elongated tubular shape and at least one wire 3 provided in the catheter body wall 2, the at least one wire 3 having at least one groove 4.

For example, the catheter 1 is not limited to a catheter that has the hub 5. The catheter body wall 2 is not limited to have the inner layer 2a and the outer layer 2b. The first wire portion 6a is not limited to a coil body. The first wire portion 6a is not limited to a radiopaque marker. The second wire portion 6b may have a structure other than a coil body and a braided body. The second wire portion 6b is not limited to a reinforcing body.

In the catheter 1 according to the above-described embodiment, it is preferable that at least one groove 4 is provided only in the distal portion 1a of the catheter 1.

In the catheter 1 according to the above-described embodiment, it is preferable that at least one groove 4 has a pitch P that decreases from the proximal side toward the distal side of the catheter 1.

In the catheter 1 according to the above-described embodiment, it is preferable that at least one groove 4 has a width W or a depth D that increases from the proximal side toward the distal side of the catheter 1.

In the catheter 1 according to the above-described embodiment, it is preferable that at least one wire 3 forms a radiopaque marker or a reinforcing body.

In the catheter 1 according to the above-described embodiment, it is preferable that at least one wire 3 is provided in the catheter body wall 2 over the entire circumference, meaning the at least one wire 3 has a circumferential extent of at least 360°.

In the catheter 1 according to the above-described embodiment, it is preferable that at least one wire 3 forms a coil body or a braided body.

In the catheter 1 according to the above-described embodiment, it is preferable that at least one groove 4 is provided in a section of at least one wire 3 extending along the entire circumference of the catheter body wall 2. That is, the at least one groove 4 is provided in a section of at least one wire 3 that has a circumferential extent of at least 360° so that the at least one groove has a circumferential extent of at least 360°.

In the catheter 1 according to the above-described embodiment, it is also preferable that at least one groove 4 is provided in a section of at least one wire 3 extending in a second direction perpendicular to both an axial direction of the catheter body wall 2 and a first direction perpendicular to the axial direction of the catheter body wall 2 without being provided in a section of at least one wire 3 extending in the first direction.

In this case, it is preferable that, in the catheter 1, at least one groove 4 is provided in only both surfaces or in only one surface of at least one wire 3 in the second direction. In addition, it is also preferable that, in the catheter 1, at least one groove 4 is provided in only both surfaces or in only one surface of at least one wire 3 in the axial direction of the catheter body wall 2.

In the catheter 1 according to the above-described embodiment, it is preferable that at least one groove 4 extends in the axial direction of the catheter body wall 2. In this case, it is preferable that, in the catheter 1, at least one groove 4 is provided only in a section of at least one wire 3 facing toward the outside the catheter body wall 2 in a radial direction.

In the catheter 1 according to the above-described embodiment, it is also preferable that at least one groove 4 is provided helically or annularly in an outer peripheral surface of at least one wire 3.

It is preferable that a method for manufacturing the catheter 1 according to the above embodiment includes forming at least one groove 4 by irradiating at least one wire 3 with a laser beam in a state where at least one wire 3 is provided in the catheter body wall 2.

The detailed description above describes embodiments of a catheter and manufacturing method representing examples of the catheter and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter comprising:
a catheter body wall having an elongated tubular shape and possessing an inner peripheral surface and an outer peripheral surface;
the catheter body wall being comprised of an inner layer and an outer layer;
the outer layer of the catheter body wall being positioned radially outwardly of the inner layer of the catheter body wall;
at least one wire in the catheter body wall, the at least one wire being positioned radially inwardly of the outer peripheral surface of the catheter body wall and radially outwardly of the inner peripheral surface of the catheter body wall; and
the at least one wire being provided with at least one groove, a part of the outer layer of the catheter body wall being located in at least a portion of the at least one groove of the at least one wire.

2. The catheter according to claim 1, wherein the at least one groove is a plurality of grooves that are spaced apart from one another along the at least one wire.

3. The catheter according to claim 1, wherein the at least one wire is a helically wound wire comprised of a plurality of axially adjacent windings, the at least one groove being a groove that extends helically around the at least one wire.

4. The catheter according to claim 1, wherein the catheter body wall having the elongated tubular shape extends in an axial direction, the at least one wire being a wire that has an axial extent that extends helically, the at least one groove including a plurality of grooves spaced apart from one another in the axial direction and extending along at least a portion of the axial extent of the at least one wire.

5. The catheter according to claim 1, wherein the at least one wire has an axial extent that extends helically, the at least one groove including a plurality of grooves spaced apart from one another along the axial extent and each having a circumferential extent less than 360°.

6. A catheter comprising:
a catheter body wall having an elongated tubular shape; and
at least one wire provided in the catheter body wall, the at least one wire having at least one groove.

7. The catheter according to claim 6, wherein the at least one groove is provided only at a distal portion of the catheter.

8. The catheter according to claim 6, wherein the at least one wire is a radiopaque marker or a reinforcing body.

9. The catheter according to claim 6, wherein the at least one wire is provided in the catheter body wall over an entire circumference of the catheter body wall.

10. The catheter according to claim 11, wherein the at least one wire is a coil body or a braided body.

11. The catheter according to claim 6, wherein the at least one groove is provided in a section of the at least one wire extending along an entire circumference of the catheter body wall so that the at least one groove extends along the entire circumference of the catheter body wall.

12. The catheter according to claim 9, wherein the at least one groove is provided in a section of the at least one wire extending in a second direction perpendicular to both an axial direction of the catheter body wall and a first direction perpendicular to the axial direction of the catheter body wall without being provided in a section of the at least one wire extending in the first direction.

13. The catheter according to claim 12, wherein the at least one groove is provided in only both surfaces or in only one surface of the at least one wire in the second direction.

14. The catheter according to claim 12, wherein the at least one groove is provided in only both surfaces or in only one surface of the at least one wire in the axial direction of the catheter body wall.

15. The catheter according to claim 6, wherein the catheter body wall extends in an axial direction, the at least one groove extending in the axial direction of the catheter body wall.

16. The catheter according to claim 15, wherein the at least one groove is provided only in a section of the at least one wire facing toward outside the catheter body wall in a radial direction.

17. The catheter according to claim 6, wherein the at least one groove is a helically extending groove or an annularly extending groove in an outer peripheral surface of the at least one wire.

18. A method for manufacturing a catheter comprising:
forming at least one groove in an outer peripheral surface of a wire that is provided in a catheter body wall of a catheter; and
the forming of the at least one groove comprising irradiating the at least one wire with a laser beam while the at least one wire is provided in the catheter body wall.

19. The method according to claim 18, wherein the at least one wire is made of a first material and the catheter body wall is made of a second material, the laser beam that irradiates the at least one wire having a wavelength that is more easily absorbed by the first material and less easily absorbed by the second material.

20. The method according to claim 18, wherein the catheter body wall is a tubular catheter body wall having an outer surface, the at least one wire being provided on the outer surface of the at least wire during the irradiation, and an outer layer being provided in covering relation to the at least one wire after the irradiation.
